(19) United States
(12) Patent Application Publication
(10) Pub. No.: US 2021/0403491 A1
(43) Pub. Date: Dec. 30, 2021

(54) CESIUM OXALATE PRODUCTION FROM CESIUM CARBONATE

(71) Applicant: SABIC Global Technologies B.V., Bergen op Zoom (NL)

(72) Inventors: Balamurugan VIDJAYACOUMAR, Thuwal (SA); Ahmad AL-JABER, Thuwal (SA); Mohammed BABKOOR, Thuwal (SA); Ilia KOROBKOV, Thuwal (SA); Khalid ALBAHILY, Thuwal (SA)

(21) Appl. No.: 16/625,339

(22) PCT Filed: Jun. 19, 2018
(86) PCT No.: PCT/IB2018/054515

§ 371 Date: Dec. 20, 2019

Related U.S. Application Data
(60) Provisional application No. 62/523,002, filed on Jun. 21, 2017.

(51) Int. Cl. C07F 9/00

(52) U.S. Cl. CPC C07F 9/00

(57) ABSTRACT

Processes for producing a disubstituted oxalate are disclosed. The process includes contacting a mixture of cesium salt and gamma alumina with one or more alcohols and carbon dioxide (CO₂) under reaction conditions sufficient to produce a composition comprising a disubstituted oxalate.

### CROSS REFERENCE TO RELATED APPLICATIONS

This application claims the benefit of priority of U.S. Provisional Patent Application No. 62/523,002 filed Jun. 21, 2017, which is hereby incorporated by reference in its entirety.

### BACKGROUND OF THE INVENTION

### A. Field of the Invention

The invention generally concerns a process for preparing cesium oxalate (Cs₂C₂O₄). In particular, the process includes contacting a gaseous reactant(s) that includes a carbon source and an oxygen source with a mixture of gamma alumina and a cesium salt under reaction conditions sufficient to produce a mixture that includes Cs₂C₂O₄. The produced Cs₂C₂O₄ can be converted into a range products, for example disubstituted oxalates (e.g., DMO), oxalic acids, oxamides, or ethylene glycol.

### B. Description of Related Art

DMO is the dimethyl ester of oxalic acid. DMO is used in various industrial processes, such as in pharmaceutical products, for the production of oxalic acid and ethylene glycol, or as a solvent or plasticizer. Commercially, DMO can be prepared by high pressure oxidative coupling of carbon monoxide and an alkyl nitrite in the presence of a palladium catalyst. These types of processes need relatively large amounts of carbon monoxide as a feedstock. Carbon monoxide is mainly produced from the gasification of coal. Due to depleting global fossil fuels reserves, there is a foreseeable demand for new processes that require alternate feedstocks for DMO production.

### SUMMARY OF THE INVENTION

A discovery has been made that provides an improved catalyst for the production of disubstituted oxalates (e.g., DMO). The improved catalyst is prepared by contacting a mixture of a cesium salt (e.g., Cs₂CO₃) and gamma alumina with a gaseous carbon feed source and a gaseous oxygen feed source to form composition that includes cesium oxalate (Cs₂C₂O₄) and gamma alumina. The gaseous carbon source and gaseous oxygen source can be derived from mixtures of carbon dioxide (CO₂) and carbon monoxide (CO) or hydrogen (H₂), or a mixture of CO and oxygen (O₂). The produced Cs₂C₂O₄ can then be selectively converted to DMO when contacted with a methanol (CH₃OH) and CO₂. Without wishing to be bound by theory, it is believed that combing the cesium salt (e.g., Cs₂CO₃) with gamma alumina can inhibit the cesium oxalate from forming a melt that requires further processing (e.g., grinding, powdering, etc.) prior to reaction with alcohol to form the disubstituted oxalate of the present invention. At least three other benefits can be obtained by this synthesis process: (1) the reliance on CO as a feed stock to produce DMO can be reduced or avoided; (2) the overall production of DMO from Cs₂CO₃ can be performed in a step-wise manner or in a single-pot fashion where Cs₂C₂O₄ is generated in situ and then converted to DMO; and/or (3) the use of expensive noble metal catalysts such as palladium-based catalysts can be reduced or avoided.

In one aspect of the present invention there is disclosed a process for preparing cesium oxalate (Cs₂C₂O₄). The process can include contacting a gaseous reactant(s) that includes a carbon source and an oxygen source with a mixture of gamma alumina and a cesium salt under reaction conditions sufficient to form a composition that includes Cs₂C₂O₄. In another aspect, the cesium salt is in contact with a surface of the gamma alumina, preferably mixed in the gamma alumina. The cesium salt can be cesium carbonate (Cs₂CO₃). The cesium salt mixture can be prepared by mixing the gamma alumina with the cesium salt (e.g., Cs₂CO₃) in a mass ratio of gamma alumina to cesium salt can be 0.1:10 to 10 to 0.1, 0.5:5, 1:1, 2:1, about 1:1, or about 0.5:1.

In one embodiment, the process can include contacting CO₂ and CO with the cesium salt mixture under reaction conditions sufficient to form a composition that includes Cs₂C₂O₄. In another embodiment, the process can include contacting CO₂ and H₂ with the cesium salt mixture under reaction conditions sufficient to form a composition comprising Cs₂C₂O₄. In still another embodiment, the process can include contacting CO₂ and CO with the cesium salt mixture under reaction conditions sufficient to form a composition comprising Cs₂C₂O₄. The reaction conditions for each embodiment can include a reaction temperature of 250° C. to 400° C., 300° C. to 375° C., preferably 310° C. to 335° C., or most preferably 320° C. to 330° C., and a reaction pressure of 1 MPa to 6 MPa, 2 MPa to 5 MPa, or preferably 3 MPa to 4 MPa. In certain aspects, the reaction conditions can include providing CO₂ at a pressure of 2.0 MPa to 4.0 MPa, preferably about 2.5 MPa, and CO at a pressure of 1 MPa to 3 MPa, preferably about 2 MPa. In other instances, cesium bicarbonate (CsHCO₃) can also be formed along with Cs₂C₂O₄. Notably, cesium formate is not formed (e.g., formed in undetectable amounts).

The produced product stream or composition that includes Cs₂C₂O₄ can be stored for later use in producing a disubstituted oxalate, an oxalic acid, an oxamide, or ethylene glycol. In some instances, the Cs₂C₂O₄ can be isolated from the product mixture and/or further purified. In other instances, the produced Cs₂C₂O₄ can be directly converted into a disubstituted oxalate, an oxalic acid, oxamide, or ethylene glycol in the same reaction procedure such as in a one-pot reaction scheme. The reaction conditions for converting the produced Cs₂C₂O₄ into a disubstituted oxalate can include contacting Cs₂C₂O₄ with one or more alcohols and additional CO₂ under conditions sufficient to produce a disubstituted oxalate, preferably DMO. Such conditions can include a reaction temperature of 100° C. to 300° C., 125° C. to 175° C., or preferably about 200° C. and/or a pressure of 2 MPa to 5 MPa, 3 MPa to 4 MPa, or preferably about 3.5 MPa. In some aspect, the alcohol can be methanol, ethanol, propanol, etc. When DMO is produced, the preferred alcohol is methanol. The process of converting the Cs₂C₂O₄ into DMO can also result in the production of methyl formate.

Also described in the context of the present invention are compositions. One composition can be used for producing cesium oxalate, the composition can include a mixture of cesium carbonate and gamma alumina. The composition can further include a gaseous reactant(s) that includes a carbon source and an oxygen source. Another composition of the present invention for producing disubstituted oxalate can include cesium carbonate, gamma alumina, carbon dioxide (CO₂), and an alcohol.

In the context of the present invention, 20 embodiments are described. Embodiment 1 is a process for preparing cesium oxalate (Cs₂C₂O₄), the process comprising contacting a gaseous reactant(s) that includes a carbon source and an oxygen source with a mixture of gamma alumina and a cesium salt under reaction conditions sufficient to form a composition comprising Cs₂C₂O₄. Embodiment 2 is the process of embodiment 1, wherein the cesium salt is in contact with a surface of the gamma alumina, preferably mixed in the gamma alumina. Embodiment 3 is the process of any one of embodiments 1 to 2, wherein the cesium salt is cesium carbonate (Cs₂CO₃). Embodiment 4 is the process of any one of embodiments 1 to 3, wherein no cesium formate is formed, or formed in undetectable amounts, under the reaction conditions. Embodiment 5 is the process of any one of embodiments 1 to 4, wherein a mass ratio of gamma alumina to the cesium salt is 0.1:10 to 10:0.1, or 0.5:5, 1:1, 2:1. Embodiment 6 is the process of embodiment 5, wherein the mass ratio is 0.5:1. Embodiment 7 is the process of any one of embodiments 1 to 6, wherein the gaseous reactants include carbon dioxide (CO₂) and carbon monoxide (CO) or hydrogen (H₂), or include CO and oxygen (O₂). Embodiment 8 is the process of embodiment 7, wherein the gaseous reactants include CO₂ and CO. Embodiment 9 is the process of any one of embodiments 1 to 8, wherein the reaction conditions comprise a temperature of 250° C. to 400° C., 300° C. to 375° C., preferably 310° C. to 335° C., or most preferably 320° C. to 330° C., a pressure of 1 MPa to 6 MPa, 2 MPa to 5 MPa, or preferably 3 MPa to 4 MPa, or combinations thereof. Embodiment 10 is the process of any one of embodiments 8 to 9, wherein the reaction conditions comprise providing CO₂ at a pressure of 2.0 MPa to 4.0 MPa, preferably about 2.5 MPa, and CO at a pressure of 1 MPa to 3 MPa, preferably about 2 MPa. Embodiment 11 is the process of any of embodiments 1 to 10, wherein cesium bicarbonate (CsHCO₃) is formed. Embodiment 12 is the process of any one of embodiments 1 to 11, further comprising isolating the Cs₂C₂O₄ from the product stream. Embodiment 13 is the process of any one of embodiments 1 to 12, further comprising converting the Cs₂C₂O₄ to a disubstituted oxalate, oxalic acid, oxamide, or ethylene glycol. Embodiment 14 is the process of any one of embodiments 1 to 13, wherein Cs₂C₂O₄ is generated in situ and then contacted with the one or more alcohols and additional CO₂ under conditions sufficient to produce a disubstituted oxalate. Embodiment 15 is the process of embodiment 14, wherein the conditions sufficient to produce a disubstituted oxalate comprise a temperature of 100° C. to 220° C., 125° C. to 210° C., or preferably about 210° C., and optionally a pressure of 2 MPa to 5 MPa, 3 MPa to 4 MPa, or preferably about 4.5 MPa. Embodiment 16 is the process of any one of embodiments 14 to 15, wherein the alcohol is methanol and the disubstituted oxalate is dimethyl oxalate (DMO). Embodiment 17 is the process of embodiment 16, wherein methyl formate is formed.

Embodiment 18 is a composition for producing cesium oxalate, the composition comprising a mixture of cesium carbonate and gamma alumina, wherein the composition further comprises a gaseous reactant(s) that includes a carbon source and an oxygen source. Embodiment 19 is the composition of embodiment 18, wherein the carbon source and the oxygen source is CO and CO₂. Embodiment 20 is a composition for producing disubstituted oxalate, the composition comprising cesium carbonate, gamma alumina, carbon dioxide (CO₂), and an alcohol.

The following includes definitions of various terms and phrases used throughout this specification.

The term “alkyl group” can be a straight or branched chain alkyl having 1 to 20 carbon atoms. Examples include methyl, ethyl, propyl, isopropyl, butyl, isobutyl, secondary butyl, tertiary butyl, pentyl, isopentyl, neopentyl, hexyl, benzyl, heptyl, octyl, 2-ethylhexyl, 1,1,3,3-tetramethylbutyl, nonyl, decyl, dodecyl, tetradecyl, hexadecyl, octadecyl, and/or eicosyl.

The term “substituted alkyl group” can include any of the aforementioned alkyl groups that are additionally substituted with one or more heteroatom, such as a halogen (F, Cl, Br, I), boron, oxygen, nitrogen, sulfur, silicon, etc. Without limitation, a substituted alkyl group can include alkoxy or alkylamine groups where the alkyl group attached to the heteroatom can also be a substituted alkyl group.

The term “aromatic group” can be any aromatic hydrocarbon group having 5 to 20 carbon atoms of the monocyclic, polycyclic or condensed polycyclic type. Examples include phenyl, biphenyl, naphthyl, and the like. Without limitation, an aromatic group also includes heteroaromatic groups, for example, pyridyl, indolyl, indazolyl, quinolinyl, isoquinolinyl, and the like.

The term “substituted aromatic group” can include any of the aforementioned aromatic groups that are additionally substituted with one or more atom, such as a halogen (F, Cl, Br, I), carbon, boron, oxygen, nitrogen, sulfur, silicon, etc. Without limitation, a substituted aromatic group can be substituted with alkyl or substituted alkyl groups including alkoxy or alkylamine groups.

The terms “about” or “approximately” are defined as being close to as understood by one of ordinary skill in the art. In one non-limiting embodiment, the terms are defined to be within 10%, preferably, within 5%, more preferably, within 1%, and most preferably, within 0.5%.

The terms “wt. %,” “vol. %,” or “mol. %” refers to a weight, volume, or molar percentage of a component, respectively, based on the total weight, the total volume, or the total moles of material that includes the component. In a non-limiting example, 10 moles of component in 100 moles of the material is 10 mol. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The process of the present invention can “comprise,” “consist essentially of,” or “consist of” particular ingredients, components, compositions, etc., disclosed throughout the specification. With respect to the transitional phase “consisting essentially of,” in one non-limiting aspect, a basic and novel characteristic of the process of the present invention is the ability to produce Cs₂C₂O₄ by contacting gamma alumina Cs₂CO₃ with an oxygen source and a carbon source (e.g., CO₂, CO, or mixtures thereof alone, and/or in combination with H₂, O₂, or mixtures thereof). In some particular instances of the present invention, the produced Cs₂C₂O₄ can then be converted to DMO in the presence of methanol.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

Advantages of the present invention may become apparent to those skilled in the art with the benefit of the following detailed description and upon reference to the accompanying drawings.

FIG. 1 is the CO to CO₂ transformation energies.

FIG. 2 is the Cs₂CO₃ to Cs₂C₂O₄ transformation energies.

FIG. 3 is the Cs₂C₂O₄ to DMO transformation energies.

FIG. 4 is a schematic of a one reactor system to produce disubstituted oxalates of the present invention.

FIG. 5 is a schematic of a two reactor system to produce disubstituted oxalates of the present invention.

While the invention is susceptible to various modifications and alternative forms, specific embodiments thereof are shown by way of example in the drawings. The drawings may not be to scale.

### DETAILED DESCRIPTION OF THF INVENTION

A discovery has been made that provides an elegant solution to some the problems of diminishing feedstocks for the production of disubstituted oxalates such as dimethyl oxalate. The discovery is premised on producing Cs₂C₂O₄ by contacting a gaseous carbon source and a gaseous oxygen source with a cesium salt (e.g., Cs₂CO₃) and gamma alumina composition. The gaseous carbon source and a gaseous oxygen source can include a combination of CO₂ and CO, CO₂ and H₂, or CO and O₂. The produced Cs₂C₂O₄ can then be selectively converted to a disubstituted oxalate such as dimethyl oxalate when contacted with one or more alcohols and CO₂ under appropriate reaction conditions. The following reaction equation (1) includes the overall general reaction for the production of disubstituted oxalates:

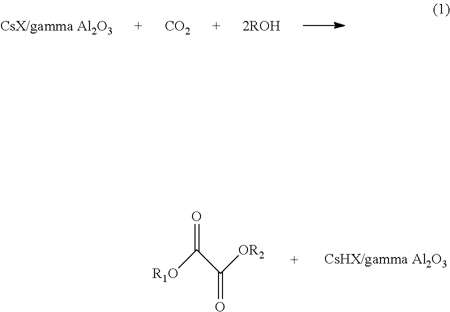

where X is a counter anion to the cesium metal cation and ROH can be the same or different alcohols and R₁ and R₂ where ROH can be the same or different alcohols and R₁ and R₂ are defined below. In a preferred embodiment, ROH is methanol and the disubstituted oxalate is dimethyl oxalate.

These and other non-limiting aspects of the present invention are discussed in further detail in the following sections with reference to the Figures.

### A. Cesium Oxalate Production

Cesium oxalate production can be produced in the context of the present invention by contacting a mixture of gamma alumina and a cesium salt (e.g., Cs₂CO₃) with an oxygen source and a carbon source under reaction conditions sufficient to form a composition that includes Cs₂C₂O₄. In some embodiments, the product composition is substantially Cs₂C₂O₄, preferably 100 mol. % Cs₂C₂O₄. The composition can also include cesium bicarbonate (CsHCO₃). Notably, cesium formate is not formed. Formation of a cesium oxalate in presence of gamma alumina can inhibit the cesium oxalate from forming a melt that requires further processing (e.g., grinding, powdering, etc.) prior to reaction with other reagents to form various products (e.g., disubstituted oxalates, oxalic acids, oxamides, or ethylene glycol), especially when the cesium oxalate is generated in situ. A mass ratio of gamma alumina to the cesium salt can be 0.1:10 to 10:0.1, or 0.2:8, 0.5:5, 1:1, 2:1, 5:0.2, or 8:0.5. In one non-limiting embodiment, the mass ratio of gamma alumina to the cesium salt can be 1:1, or 0.5:1. The gamma alumina can have a surface area of 250 to 260 m²/g (e.g., 250, 251, 252, 253, 254, 255, 256, 257, 258, 259, 260 m²/g or any value or range there between) and have a bimodal pore distribution. The oxygen source and the carbon source can be obtained from one or more compounds. Non-limiting examples of gaseous reactants that include a carbon source and an oxygen source can include (i) CO₂ and CO, (ii) CO₂ and H₂, or (iii) CO and O₂. In a non-limiting embodiment, the gaseous reactants can include CO₂ and CO.

Reaction conditions to produce the cesium oxalate can include temperature and/or pressure. Non-limiting examples of a reaction temperature include temperatures from 250° C. to 400° C., 300° C. to 375° C., preferably 310° C. to 335° C., or most preferably 320° C. to 330° C. or at least equal to, greater than or between any two 250, 260, 270, 280, 290, 300, 310, 320, 330, 340, 350, 360, 370, 380, 390, and 400° C. Non-limiting examples of a reaction pressure include pressures from 1 MPa to 6 MPa, 2 MPa to 5 MPa, or preferably 3 MPa to 4 MPa, or at least equal to, greater than or between any two of 1, 2, 3, 4, 5, and 6 MPa. In certain aspects, the reaction conditions can include providing CO₂ at a pressure of 2.0 MPa to 4.0 MPa, preferably about 2.5 MPa, and CO at a pressure of 1 MPa to 3 MPa, preferably about 2 MPa.

In some embodiments, cesium oxalate is generated by the reaction of cesium carbonate with carbon monoxide and carbon dioxide in the presence of a gamma alumina as shown in reaction equation (2).

Cs₂CO₃/gamma alumina+CO+CO₂→Cs₂(C₂O₄)/gamma alumina (2).

In another embodiment of the present invention, cesium oxalate can be generated by reacting cesium carbonate/gamma alumina with carbon dioxide and H₂ as shown in reaction equation (3) and as described in more detail below and in the Examples section.

Cs₂CO₃/gamma alumina+H₂+CO₂→Cs₂(C₂O₄)/gamma alumina (3).

In some embodiments, the carbon dioxide and H₂ are added in a sequential manner as shown in reaction equation (4). The sequential addition of carbon dioxide then hydrogen in the presence of gamma alumina can inhibit or substantially inhibit the formation of cesium formate (HCO₂Cs). Limiting the formation of cesium formate can limit the formation of alkyl formate in subsequent reactions with alcohols. In some instances, cesium formate is not formed in the production of cesium oxalate.

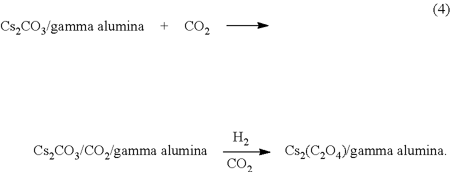

In yet another alternative process, the cesium oxalate can be generated by the reaction of cesium carbonate with carbon monoxide and O₂ as shown in reaction equation (5) as described in more detail below.

Cs₂CO₃/gamma alumina+CO+O₂→Cs₂(C₂O₄)/gamma alumina (5).

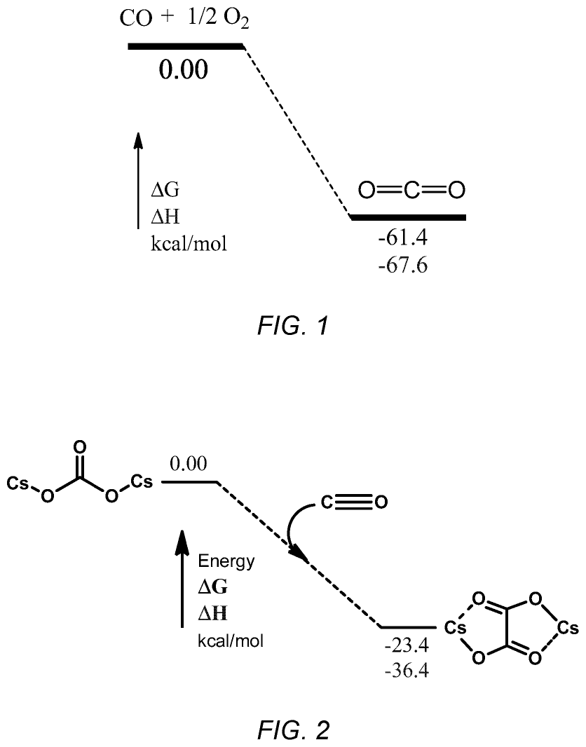

With respect to reaction equation (5), and without wishing to be bound by theory, it is believed that the use of molecular oxygen will require lower heat requirements when compared to the other processes as the reaction between CO and O₂ is exothermic (free energy change of −61.4 kcal/mol as determined through density functional theory (DFT)). FIG. 1 depicts the carbon monoxide to carbon dioxide transformation energetics. The CO₂ can bind with cesium carbonate to form a CO₂—Cs₂CO₃ adduct, which has an enthalphy of fusion as at molecular level. This enthalpy of fusion can be compensated by the CO+0.5 O₂ to CO₂ energy of 122.8 kcal/mol. The remaining carbon monoxide can then transform CO₂—Cs₂CO₃ adduct into cesium oxalate. FIG. 2 shows the overall Cs₂CO₃ to Cs₂C₂O₄ transformation energies. Thus, overall reaction equation (5) is exothermic with a calculated free energy (DFT) change of −23.4 kcal/mol making the reaction favorable for low heating requirements.

### B. Disubstituted Oxalate

The produced cesium oxalate/gamma alumina product from Section A can then be reacted with a desired alcohol in the presence of carbon dioxide to produce a desired disubstituted oxalate. In some instances, the produced cesium oxalate product is first purified before being converted to a disubstituted oxalate. Such purification may help with reducing or avoiding the formation of undesired by-products during disubstituted oxalate production. Reaction equations (7) through (10) show the overall reaction starting with a mixture of gamma alumina and cesium salt (CsX), preferably a mixture of cesium carbonate and gamma alumina. Reaction conditions are described in more detail below and in the Examples Section. In the reactions, ROH can be any alcohol or a mixture of alcohols, preferably methanol, and Al₂O₃ is gamma-Al₂O₃.

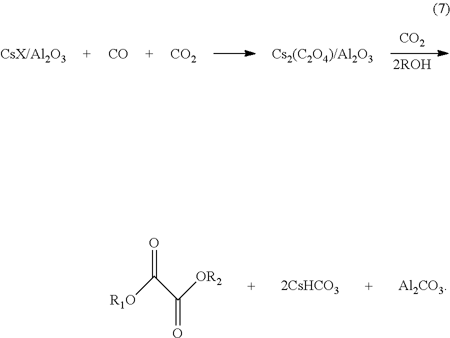

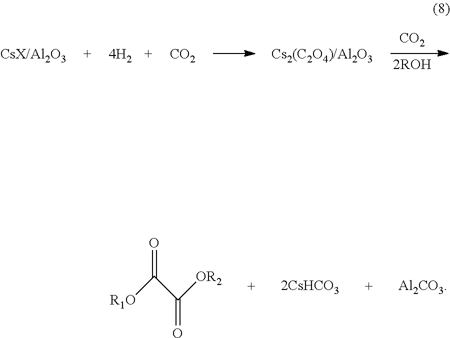

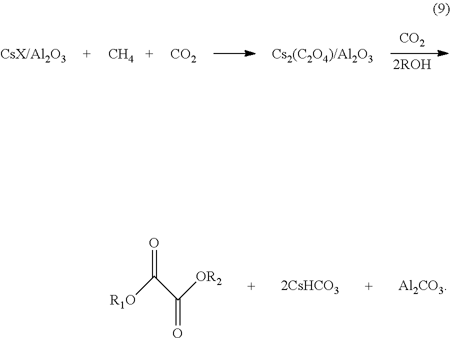

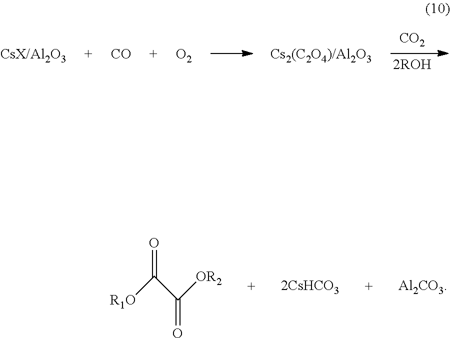

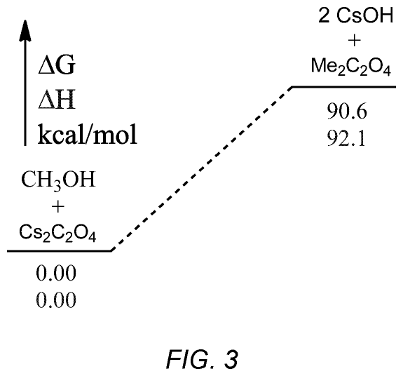

Without wishing to be bound by theory, it is believed that the conversion of cesium oxalate to disubstituted oxalates (e.g., dimethyl oxalate (DMO)) is endothermic with an overall calculated free energy (DFT) change of about 91 kcal/mol. For example, FIG. 3 shows Cs₂C₂O₄ to DMO transformation energies. Thus, the exothermic formation of cesium oxalate from cesium carbon monoxide and oxygen illustrated in reaction equation (9) can provide energy for this step, thereby requiring less overall energy (e.g., heat input).

### C. Sustainability

Under certain conditions, cesium bicarbonate can be formed from the cesium bicarbonate (e.g., 2CsHCO₃ yields Cs₂CO₃+CO₂+H₂O). Cesium bicarbonate products can be separated or further processed. Notably, cesium hydroxide is not formed or formed in undetectable amounts. The overall sustainable process is shown in the schematic below. As discussed above and throughout this specification, the combination of “reactant 1” and “reactant 2” in the schematic can be a combination of CO₂+CO, CO₂+H₂, or CO+O₂.

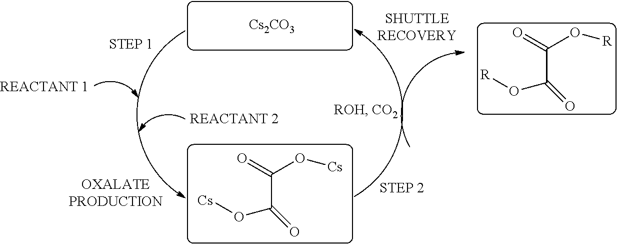

### D. System and Processes to Prepare Cesium Oxalate and Disubstituted Oxalate

1. Single Reactor Preparation of Cesium Oxalate and Disubstituted Oxalate

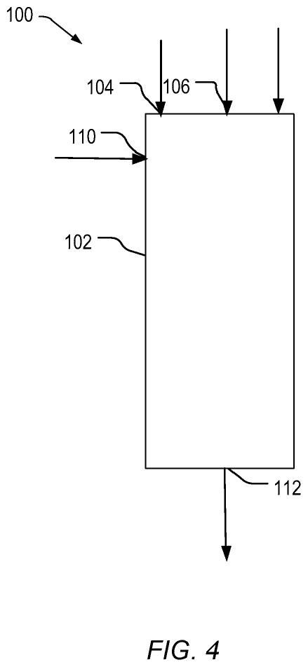

Any of the processes of the present invention can be performed in a single reactor. Referring to FIG. 4, a method and system to prepare disubstituted oxalates is described. In system 100, a mixture of gamma alumina and cesium salt (e.g., cesium carbonate (Cs₂CO₃)) can be provided to reactor unit 102 via solids inlet 104. In some embodiments, the gamma alumina and cesium salt are provided to the reactor unit 102 and mixed in the reactor unit. An oxygen and carbon source (e.g., CO, CO₂, O₂, or H₂ or any oxygen/carbon combination thereof) can be provided to reactor 102 via gas inlets 106 and 108. By way of example, CO₂ can be provided to reactor 102 via gas inlet 106 and CO via gas inlet 108. In another embodiments, the mixture of cesium salt and gamma alumina and be added to reactor unit 104 and CO₂ can be provided to reactor 102 via gas inlet 106. The CO₂ can be provided to reactor 102 at a pressure ranging from 1 MPa to 3 MPa and all ranges and pressures there between (e.g., 1.1 MPa, 1.2 MPa, 1.3 MPa, 1.4 MPa, 1.5 MPa, 1.6 MPa, 1.7 MPa, 1.8 MPa, 1.9 MPa, 2 MPa, 2.1 MPa, 2.2 MPa, 2.3 MPa, 2.4 MPa, 2.5 MPa, 2.6 MPa, 2.7 MPa, 2.8 MPa, or 2.9 MPa, preferably 2.5 MPa). The mixture can be heated under CO₂ atmosphere to about 300° C. to 350° C. or any range or value there between (e.g., 305° C., 310° C., 315° C., 320° C., 325° C., 330° C., 335° C., 340° C., 345° C., 350° C., preferably about 325° C. for about 0.5 to 24 hours, preferably about 1 hour. After the heating period, reactor 102 can be cooled to a temperature sufficient to allow CO to be added to reactor 102 via gas inlet 108. The CO can be provided to reactor 102 at a pressure ranging from 1 MPa to 3 MPa and all ranges and pressures there between (e.g., 1.1 MPa, 1.2 MPa, 1.3 MPa, 1.4 MPa, 1.5 MPa, 1.6 MPa, 1.7 MPa, 1.8 MPa, 1.9 MPa, 2 MPa, 2.1 MPa, 2.2 MPa, 2.3 MPa, 2.4 MPa, 2.5 MPa, 2.6 MPa, 2.7 MPa, 2.8 MPa, or 2.9 MPa). Preferably, the CO pressure is about 2 MPa. After CO addition, reactor 102 can be heated to a desired reaction temperature as described below.

In some embodiments, CO₂ can be provided to reactor 102 via gas inlet 106 and H₂ via gas inlet 108. Even further, CO can be provided via gas inlet 106 and O₂ via gas inlet 108. Alternatively, CO₂ and H₂ or CO and O₂, can be provided to the reactor 102 via gas inlet 106 as mixtures (e.g., a mixture of CO₂ and H₂ or a mixture of CO and O₂). In embodiments when carbon monoxide is used, the CO can be provided to reactor 102 at a pressure ranging from 1 MPa to 3 MPa and all ranges and pressures there between (e.g., 1.1 MPa, 1.2 MPa, 1.3 MPa, 1.4 MPa, 1.5 MPa, 1.6 MPa, 1.7 MPa, 1.8 MPa, 1.9 MPa, 2 MPa, 2.1 MPa, 2.2 MPa, 2.3 MPa, 2.4 MPa, 2.5 MPa, 2.6 MPa, 2.7 MPa, 2.8 MPa, or 2.9 MPa). Preferably, the CO pressure is about 2 MPa. In other embodiments when H₂ is used, the H₂ can be provided to reactor 102 at a pressure ranging from 0.05 MPa to 0.5 MPa, 0.05 to 0.4 MPa, 0.05 to 0.3 MPa, 0.05 to 0.2 MPa, or 0.05 to 0.1 and all ranges and pressures there between (e.g., 0.05 MPa, 0.1 MPa, 0.15 MPa, 0.20 MPa, 0.25 MPa, 0.30 MPa, 00.35 MPa, 0.40 MPa, 0.45 MPa, or 0.50 MPa). Preferably, the H₂ pressure is about 0.1 MPa. In other embodiments when O₂ is used, the O₂ can be provided to reactor 102 at a pressure ranging from 0.1 MPa to 5 MPa, 0.5 to 1.5 MPa, or about 0.2 MPa. CO₂ can be provided to reactor 102 at a pressure ranging from 1 MPa to 4 MPa and all ranges and pressures there between (e.g., 1.1 MPa, 1.5 MPa, 2 MPa, 2.5 MPa, 3.0 MPa, 3.5 MPa, or 4 MPa). Preferably, the CO₂ pressure is about 2.5 MPa to 3.5 MPa. The upper limit on pressure can be determined by the type and size of reactor used. Although not shown, in some embodiments, CO₂CO, O₂, or H₂, and can be provided to reactor unit 102 via the same inlet. In certain embodiments, mixtures of CO₂, CO, O₂, and H₂ are used. Reactor 102 can be pressurized either through the addition of the gases and/or with an inert gas. The average pressure of reactor unit 102 can range from 2.0 to 4 MPa (e.g., 2.0, 2.5, 3.0, 3.5, 3.9, or 4 MPa) after charging the CO₂.

After charging the gases to reactor 102, the reactor can be heated to a temperature sufficient to promote the reaction of cesium carbonate with CO₂ and CO, CO₂ and H₂, or with CO and O₂, to produce a product composition that includes cesium oxalate. The temperature range of the reactor 102 can be 200° C. to 400° C., 250° C. to 350° C., and all ranges and temperatures there between (e.g., 210° C., 220° C., 230° C., 240° C., 250° C., 260° C., 270° C., 280° C., 290° C., 300° C., 310° C., 320° C., 330° C., 340° C., 350° C., 360° C., 370° C., 380° C., or 390° C.). Preferably, the reaction temperature is 290° C. to 335° C., or 300° C. to 325° C. The reactants can be heated for a time sufficient to react all or a substantially all of the cesium carbonate. By way of example, the reaction time range can be at least 1 hour, 1 to 5 hours, 1 hours to 4 hours, 1 hour to 3 hours, and all ranges and times there between (e.g., 1 hour, 1.25 hours, 1.5 hours, 1.75 hours, 2 hour, 2.25 hours, 2.5 hours, 2.75 hours, 3 hours, 3.25 hours, 3.5 hours, 3.75 hours, 4 hours, 4.25 hours, 4.5 hours, 4.75 hours, or 5 hours). When CO₂ and CO are used, the reaction time can be about 1 to 3 hours, or preferably about 2 hours. When CO and O₂ are used, the reaction time can be about 1 to 3 hours, or preferably about 2 hours. When H₂ is used, the cesium carbonate can be reacted with the carbon dioxide for 1 to 3 hours, (e.g., 1, 1.5, 2, 2.5, or 3 hours), and then with H₂ for an additional 1 to 3 hours, (e.g., 1, 1.5, 2, 2.5, or 3 hours).

Reactor 102 can be cooled and/or depressurized to a temperature and pressure sufficient to add the desired alcohol. By way of example, reactor 102 can be cooled to a temperature range of 100° C. to 160° C., or 130° C. to 150° C., or about 150° C. at a pressure of 0.101 MPa to 1 MPa. The desired alcohol (e.g., methanol) can be added to reactor 102 via liquid inlet 110 to form a composition that includes a cesium salt (e.g., cesium oxalate, and optionally, cesium carbonate and/or cesium bicarbonate), an alcohol, carbon dioxide, and, optionally, carbon monoxide. The reactor can be pressurized with carbon dioxide and/or an inert gas to a pressure ranging from 2 MPa to 5 MPa, 3 MPa to 4 MPa, and all ranges and pressures there between (e.g., 2.1 MPa, 2.2 MPa, 2.3 MPa, 2.4 MPa, 2.5 MPa, 2.6 MPa, 2.7 MPa, 2.8 MPa, 2.9 MPa, 3 MPa, 3.1 MPa, 30.2 MPa, 3.3 MPa, 3.4 MPa, 3.5 MPa, 3.6 MPa, 3.7 MPa, 3.8 MPa, 3.9 MPa, 4.0 MPa, 4.1 MPa, 4.2 MPa, 4.3 MPa, 4.4 MPa, 4.5 MPa, 4.6 MPa, 4.7 MPa, 4.8 MPa, or 4.9 MPa). In some embodiments, carbon dioxide is present in sufficient amounts that additional CO₂ is not necessary.

After the addition of the alcohol, and, optionally, CO₂, the reactor can be heated to a reaction temperature sufficient to promote the cesium oxalate salt to react with the alcohol under the carbon dioxide atmosphere to produce a disubstituted oxalate containing composition. In other embodiments, sufficient carbon dioxide remains in reactor 102. The reaction temperature can be 125° C. to 200° C., 130° C. to 180° C., and all ranges and temperatures there between (e.g., 130° C., 135° C., 140° C., 145° C., 150° C., 155° C., 160° C., 165° C., 170° C., 175° C., 180° C., 185° C., 190° C., or 195° C.). Preferably, the reaction temperature is about 150° C. Reactor 102 can be heated for a time sufficient to react all or substantially all of the cesium salt (e.g., cesium oxalate). By way of example, the reaction time range can be less than 1 hour, 1 hours to 18 hours, 10 hour to 14 hours, 1 to 6 hours or 1 to 2 hours, and all ranges and times there between (e.g., 2 hours, 5 hours, 10 hours, 12 hours, 15 hours, or 17 hours). Preferably, the reaction time is 1 to 18 hours, or 15 hours. The upper limit on temperature, pressure, and/or time can be determined by the reactor used. The disubstituted oxalate reaction conditions can be further varied based on the type of the reactor used.

Reactor 102 can be cooled and depressurized to a temperature and pressure sufficient (e.g., below 50° C. at 0.101 MPa) to allow removal of the product composition containing disubstituted oxalate via product outlet 112. The product composition can be collected for further use. In some instances, the product composition can include cesium bicarbonate (CsHCO₃).

2. Two Reactors

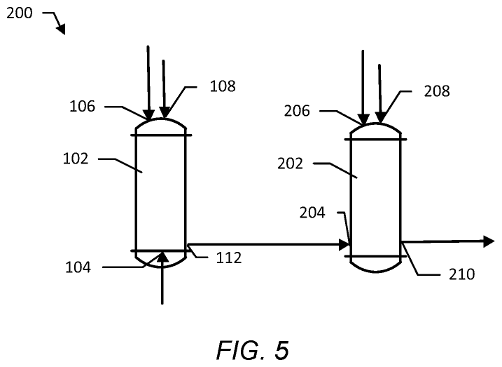

In some embodiments, reactor 102 can be depressurized and cooled to a temperature sufficient to allow the cesium oxalate containing product composition to be removed from the reactor via product outlet 112. The product composition can be further treated (e.g., washed) to remove any unreacted products. In one embodiment, the product composition is used without purification. The cesium oxalate can then be transferred to a second reactor unit to produce disubstituted oxalates. Referring to FIG. 5, a schematic of system 200 having two reactor units is depicted. The cesium salt precursor (e.g., cesium carbonate) can be provided to reactor 102 via inlet 104 and contacted with CO₂ in combination with CO, CO₂ in combination with H₂, or CO in combination with O₂ as described above (See, FIG. 1) to generate the cesium oxalate.

The cesium oxalate can exit reactor 102 via product outlet 112 and enter reactor 202 via cesium oxalate inlet 204. The desired alcohol can be provided to reactor 202 via alcohol inlet 206. Carbon dioxide can be provided to reactor 208 via carbon dioxide inlet 208. Reactor 202 can be pressurized to a pressure of 2.0 to 5 MPa (e.g., 2.0, 2.1, 2.2, 2.3, 2.4, 2.5, 3.0, 3.5, 4.0, 4.5, 5.0 MPa) either by the addition of the carbon dioxide or using an inert gas (i.e. a gas that is chemically unreactive in the present invention). Once reactor 202 has been pressurized, heat can be applied to the reactor using known methods (e.g., electrical heaters, heat transfer medium, or the like) to a temperature sufficient to promote the reaction of cesium oxalate and the alcohol. The reaction temperature can be 125° C. to 200° C., 130° C. to 180° C., and all ranges and temperatures there between (e.g., 130° C., 135° C., 140° C., 145° C., 150° C., 155° C., 160° C., 165° C., 170° C., 175° C., 180° C., 185° C., 190° C., or 195° C.). Preferably, the reaction temperature is about 150° C. Reactor 202 can be heated for a time sufficient to react all or substantially all of the cesium salt (e.g., cesium oxalate). By way of example, the reaction time range can be at least 1 hour, or 1 to 18 hours, 1 hour to 16 hours, 10 hour to 14 hours, and all ranges and times there between as previously described. Preferably, the reaction time is about 1 hour to 18 hours, or 15 hours. The upper limit on temperature, pressure, and/or time can be determined by the reactor used. The disubstituted oxalate reaction conditions can be further varied based on the type of the reactor used.

Reactor 202 can be cooled and depressurized to a temperature and pressure sufficient (e.g., below 50° C. at 0.101 MPa) to allow removal of the product composition containing disubstituted oxalate (e.g., DMO) via product outlet 210. The product composition can be collected for further use or commercial sale.

Reactors 102 and 202 and associated equipment (e.g., piping) can be made of materials that are corrosion and/or oxidation resistant. By way of example, the reactor can be lined with, or made from, Inconel. The design and size of the reactor is sufficient to withstand the temperatures and pressures of the reaction. The systems can include various automated and/or manual controllers, valves, heat exchangers, gauges, etc., for the operation of the reactor, inlets and outlets. The reactor can have insulation and/or heat exchangers to heat or cool the reactor as desired. Non-limiting examples of a heating/cooling source can be a temperature controlled furnace or an external, electrical heating block, heating coils, or a heat exchanger. The reaction can be performed under inert conditions such that the concentration of oxygen (O₂) gas in the reaction is low or virtually absent in the reaction such that O₂ has a negligible effect on reaction performance (i.e., conversion, yield, efficiency, etc.).

### E. Reactants and Products

CO₂ gas, CO gas, O₂ gas, and H₂ gas can be obtained from various sources. In one non-limiting instance, the CO₂ can be obtained from a waste or recycle gas stream (e.g., from a plant on the same site such as from ammonia synthesis, or a reverse water gas shift reaction) or after recovering the carbon dioxide from a gas stream. A benefit of recycling carbon dioxide as a starting material in the process of the invention is that it can reduce the amount of carbon dioxide emitted to the atmosphere (e.g., from a chemical production site). The CO can be obtained from various sources, including streams coming from other chemical processes, like partial oxidation of carbon-containing compounds, iron smelting, photochemical process, syngas production, reforming reactions, and various forms of combustion. O₂ can come from various sources, including streams from water-splitting reactions, or cryogenic separation systems. The hydrogen may be from various sources, including streams coming from other chemical processes, like water splitting (e.g., photocatalysis, electrolysis, or the like), syngas production, ethane cracking, methanol synthesis, or conversion of methane to aromatics. In some embodiments, the gases are obtained from commercial gas suppliers. When a mixture of gases is used to prepare cesium oxalate, for example, mixtures of CO₂ and H₂ or CO and O₂, the gas can be premixed or mixed when added separately to the reactor. When the reactor contains a mixture of CO₂ and CO, the pressure ratio of CO₂:CO in the reactor can be greater than 0.1. In some embodiments, the CO₂:CO pressure ratio can be from 0.2:1 to 5:1, from 0.5:1 to 2:1, or 1:1 to 1.5:1. Preferably, the CO₂:CO pressure ratio is about 1.25. The partial pressure of CO₂:CO in the reactor can range from 4.5 MPa to 2 MPa. When the reactor contains a mixture of CO₂ and H₂, the pressure ratio of CO₂:H₂ in the reactor can be greater than 0.1. In some embodiments, the CO₂:H₂ pressure ratio can be from 5:1 to 80:1, from 10:1 to 60:1, 20:1 to 50:1, or 30:1 to 40:1, or 35:1. Preferably, the mole CO₂:H₂ ratio is about 35:1. The partial pressure CO₂:H₂ in the reactor can range from 3.5 MPa to 1 MPa. When the reactor contains a mixture of CO and O₂, the pressure ratio of CO:O₂ in the reactor can be greater than 0.1. In some embodiments, the CO:O₂ pressure ratio can be from 5:1 to 80:1, from 10:1 to 60:1, 20:1 to 50:1, or 30:1 to 40:1, or 35:1. Preferably, the CO:O₂ pressure ratio is about 35:1. In one example, cesium carbonate is contacted with CO₂ and H₂ to form cesium oxalate. The mole ratio of CO₂ and H₂ to cesium carbonate can be 100:1 to 300:1, preferably 150:1 to 250:1, or more preferably about 200:1 and all ranges and values there between. In another example, cesium carbonate is contacted with CO and O₂ to form cesium oxalate. The mole ratio of CO and O₂ to cesium carbonate can be 1:0.1 to 3:1 and all ranges and values there between (e.g., 1:0.5, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.6, 1:2.7, 1:2.8, or 1:2.9) Preferably the ratio is 2:1. In some examples, the remainder of the reactant gas can include another gas or gases provided the gas or gases are inert, such as argon (Ar) and/or nitrogen (N₂), further provided that they do not negatively affect the reaction. Preferably, the reactant mixture is highly pure and substantially devoid of water. In some embodiments, the gases can be dried prior to use (e.g., pass through a drying media) or contain a minimal amount of water or no water at all. Water can be removed from the reactant gases with any suitable method known in the art (e.g., condensation, liquid/gas separation, etc.).

Alcohols may be purchased in various grades from commercial sources. Non-limiting examples of the alcohol that can be used in the process of the current invention to form a disubstituted oxalate can include methanol, ethanol, n-propanol, isopropanol, n-butanol, isobutanol, sec-butanol, tert-butanol, 1-pentanol, 2-pentanol, 3-pentanol, 3-methyl-1-butanol, 2-methyl-1-butanol, 2,2-dimethyl-1-propanol, 3-methyl-2-butanol, 2-methyl-2-butanol, 1-hexanol, 2-hexanol, 3-hexanol, 1-heptanol, 2-heptanol, 3-heptanol, 4-heptanol, 1-octanol, 2-octanol, 3-octanol, 4-octanol, cyclohexanol, cyclopentanol, phenol, benzyl alcohol, ethylene glycol, propylene glycol, or butylene glycol or any combination thereof. In certain embodiments, the alcohol includes a mixture of stereoisomers, such as enantiomers and diastereomers. Preferably, the alcohol is methanol, ethanol, n-propanol, isopropanol, n-butanol, isobutanol, sec-butanol, tert-butanol, 1-pentanol, 2,2-dimethyl-1-propanol (neopentanol), hexanol, or combinations thereof. When DMO is produced, the preferred alcohol is methanol.

The process of the present invention can produce a product stream that includes a composition containing a disubstituted oxalate and optionally cesium bicarbonate (CsHCO₃) that can be suitable as an intermediate or as feed material in a subsequent synthesis reactions to form a chemical product or a plurality of chemical products (e.g., such as in pharmaceutical products, for the production of oxalic acid and ethylene glycol, or as a solvent or plasticizer). In some instances, the composition containing a disubstituted oxalate can be directly reacted under conditions sufficient to form oxalic acid or ethylene glycol. The product composition can include at least 50 wt. %, at least 60 wt. %, at least 70 wt. %, at least 80 wt. %, at least 90 wt. % or 100 wt. % disubstituted oxalate, with the balance being cesium bicarbonate. The product composition can be purified using known organic purification methods (e.g., extraction, crystallization, distillation washing, etc.) depending on the phase of the production composition (e.g., solid or liquid). In a preferred embodiment, the disubstituted oxalate can be recrystallized from hot alcohol (e.g., methanol) solution. DMO can be purified by distillation (boiling point of 166° C.) or crystallization (melting point 54° C.).

The disubstituted oxalate produced by the process of the present invention can have the general structure of:

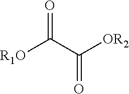

where R₁ and R₂ can be each independently alkyl group, a substituted alkyl group, an aromatic group, a substituted aromatic group, or a combination thereof. R₁ and R₂ can include 1 to 20 carbon atoms, 1 to 10 carbon atoms, 1 to 5 carbon atoms, preferably 1 carbon atom. Non-limiting examples of R₁ and R₂ include methyl, ethyl, n-propyl, isopropyl, n-butyl, isobutyl, sec-butyl, tert-butyl, 1-pentyl, 2-pentyl, 3-pentyl, 3-methyl-1-butyl, 2-methyl-1-butyl, 2,2-dimethyl-1-propyl, 3-methyl-2-butyl, 2-methyl-2-butyl, 1-hexyl, 2-hexyl, 3-hexyl, 1-heptyl, 2-heptyl, 3-heptyl, 4-heptyl, 1-octyl, 2-octyl, 3-octyl, 4-octyl, cyclohexyl, cyclopentyl, phenyl, or benzyl. Preferably, R₁ and R₂ are a methyl group, an ethyl group, a propyl group, an isopropyl group, a n-butyl group, a sec-butyl group, tert-butyl group, a pentyl group, a neopentyl, a hexyl group, or combinations thereof. In certain embodiments, R₁ and R₂ can include a mixture of stereoisomers, such as enantiomers and diastereomers. In a specific embodiment, the disubstituted oxalate is a dialkyl oxalate, such as dimethyl oxalate (DMO) where R₁ and R₂ are each methyl groups.

### EXAMPLES

The present invention will be described in greater detail by way of specific examples. The following examples are offered for illustrative purposes only, and are not intended to limit the invention in any manner. Those of skill in the art will readily recognize a variety of noncritical parameters, which can be changed or modified to yield essentially the same results.

Cesium carbonate (Cs₂CO₃) was obtained from Sigma-Aldrich® (U.S.A.) in powder form and 99.9% purity. Gamma Alumina was obtained from Alfa Aesar (U.S.A.) in powder form. The gamma alumina had a surface area of 274 m²/g, total pore volume of 1.08 cm³ g, median pore diameter of 109 Å, a particle density of 0.65 g/cm³. Methanol was obtained from Fisher Scientific (HPLC grade, U.S.A.) in 99.99% purity. ¹³C NMR was performed on a 400 MHz Bruker instrument (Bruker, U.S.A.). The Parr reactor used was obtained from Parr Instrument Company, USA.

### Example 1

### (One-Step Process for the Preparation of Dimethyl Oxalate with CO₂, CO, and Cs₂CO₃/Gamma Al₂O₃)

Gamma alumina was dried in a vacuum oven overnight at 175° C. A 1:0.5 mass ratio of Cs₂CO₃ (0.5 g) and gamma alumina (0.5 g) were placed in a high pressure reactor (100 mL Parr reactor (Parr Instrument Company, USA)) under inert conditions. CO₂ (25 bar, 2.5 MPa) was charged and the reactor heated to 325° C., maintained at 325° C. and cooled to room temperature. CO (20 bar, 2 MPa) was then charged and the mixture was stirred for 1-2 hour at 325° C. and then cooled 25° C. and depressurized. The reaction mixture contained cesium oxalate. Methanol (20 mL) was then added to the reactor, and the reactor was pressurized with CO₂ (35 bar, 3.5 MPa). The mixture was heated to 150° C., stirred overnight, and then depressurized. The remaining solvent (methanol) was removed by evaporation under vacuum. The product composition was analyzed and identified as being a mixture of dimethyl oxalate, and cesium bicarbonate. The overall yield of DMO was 97%, respectively. ¹³C NMR (CD₃OD, in ppm) of both cases: 53 (—OMe) and 158 (—CO—), 161 (CsHCO₃), and 171 (CsHCOO).

### Example 2

### (Two-Step Process for the Preparation of Dimethyl Oxalate with CO₂ and Cs₂CO₃Cs₂CO₃/Gamma Al₂O₃)

Gamma alumina was dried in vacuum oven overnight at 175° C. A 1:0.5 mass ratio of Cs₂CO₃ (1 g) and alumina (0.5 g) were placed in a 100 mL Parr reactor in the glove box. CO₂ (25 bar, 2.5 MPa) was charged and the reactor heated to 325° C., maintained at 325° C. and cooled to room temperature. CO (20 bar, 2 MPa) was were then charged and the mixture was stirred for 1-2 hour at 325° C. and then cooled to room temperature (about 25° C.) and depressurized. The reaction mixture contained cesium oxalate and was removed from the reactor. A solution of methanol (20 mL) and the crude cesium oxalate was add to the reactor, and the reactor was pressurized with CO₂ (35 bar, 3.5 MPa). The mixture was heated to 150° C., stirred overnight, and then depressurized. The remaining solvent (methanol) was removed by evaporation under vacuum. The product composition was analyzed and identified as being a mixture of dimethyl oxalate and cesium bicarbonate. The overall yield of DMO was 97%. ¹³C NMR (CD₃OD, in ppm): 53 (—OMe), 158 (—CO—), 161 (CsHCO₃), and 171 (CsHCOO).

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein can be utilized. Accordingly, the appended claims are intended to include within their scope such processes, machines, manufacture, compositions of matter, means, methods, or steps.

1. A process for preparing cesium oxalate (Cs₂C₂O₄), the process comprising contacting a gaseous reactant(s) that includes a carbon source and an oxygen source with a mixture of gamma alumina and a cesium salt under reaction conditions sufficient to form a composition comprising Cs₂C₂O₄.

2. The process of claim 1, wherein the cesium salt is in contact with a surface of the gamma alumina.

3. The process of claim 1, wherein the cesium salt is cesium carbonate (Cs₂CO₃).

4. The process of claim 1, wherein no cesium formate is formed under the reaction conditions.

5. The process of claim 1, wherein a mass ratio of gamma alumina to the cesium salt is 0.1:10 to 10:0.1, or 0.5:5, 1:1, 2:1.

6. The process of claim 5, wherein the mass ratio is 0.5:1.

7. The process of claim 1, wherein the gaseous reactants include carbon dioxide (CO₂) and carbon monoxide (CO) or hydrogen (H₂), or include CO and oxygen (O₂).

8. The process of claim 7, wherein the gaseous reactants include CO₂ and CO.

9. The process of claim 1, wherein the reaction conditions comprise a temperature of 250° C. to 400° C., 300° C. to 375° C. a pressure of 1 MPa to 6 MPa or combinations thereof.

10. The process of claim 8, wherein the reaction conditions comprise providing CO₂ at a pressure of 2.0 MPa to 4.0 MPa.

11. The process of claim 1, wherein cesium bicarbonate (CsHCO₃) is formed.

12. The process of claim 1, further comprising isolating the Cs₂C₂O₄ from the product stream.

13. The process claim 1, further comprising converting the Cs₂C₂O₄ to a disubstituted oxalate, oxalic acid, oxamide, or ethylene glycol.

14. The process of claim 1, wherein Cs₂C₂O₄ is generated in situ and then contacted with the one or more alcohols and additional CO₂ under conditions sufficient to produce a disubstituted oxalate.

15. The process of claim 14, wherein the conditions sufficient to produce a disubstituted oxalate comprise a temperature of 100° C. to 220° C. and a pressure of 2 MPa to 5 MPa.

16. The process of claim 14, wherein the alcohol is methanol and the disubstituted oxalate is dimethyl oxalate (DMO).

17. The process of claim 16, wherein methyl formate is formed.

18. A composition for producing cesium oxalate, the composition comprising a mixture of cesium carbonate and gamma alumina, wherein the composition further comprises a gaseous reactant(s) that includes a carbon source and an oxygen source.

19. The composition of claim 18, wherein the carbon source and the oxygen source is CO and CO₂.

20. A composition for producing disubstituted oxalate, the composition comprising cesium carbonate, gamma alumina, carbon dioxide (CO₂), and an alcohol.
